(19) United States
(12) Patent Application Publication
(10) Pub. No.: US 2003/0216697 A1
(43) Pub. Date: Nov. 20, 2003

(54) Device for guiding a drive in an injection device

(76) Inventor: Thomas Hegewald, Baiersdorf (DE)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/438,355

(22) Filed: May 14, 2003

(30) Foreign Application Priority Data
May 15, 2002 (DE) 10221782.3-44

(51) Int. Cl. A61M 5/315

(52) U.S. Cl. 604/218

(57) ABSTRACT

The present invention relates to a device for guiding a rod-shaped output member of an injection device with an exchangeable cartridge containing injection liquid, where the rod-shaped output member can be driven by defined distances in a direction reducing the volume of the cartridge in order to dispense the injection liquid, a clamp element being provided which can be secured releasably on the rod-shaped output member, which clamp element, in the clamped state of the rod-shaped output member, is movable in order to set the dose to be injected or to dispense the dose, and which clamp element is automatically released upon release and removal of the cartridge.

The invention relates to a device for guiding a rod-shaped output member of an device.

The Applicant markets an injection device under the name OptiPen® for insulin injection. This injection device contains a cartridge with insulin. This cartridge is exchangeable. In the injection device, the insulin is dispensed from the cartridge by means of a plunger reducing the volume of the cartridge. On the other side of the cartridge, a membrane forming part of the surface of the cartridge is pierced by the injection needle. Upon reduction of the volume, the insulin is thus dispensed through the injection needle. When the cartridge is to be changed, the plunger is located in the farthest forward position because the cartridge situated in the device has been at least almost completely emptied. However, in order to be able to insert a new, full cartridge, the plunger has to be moved back to the rearmost position. The cartridge is secured in the injection device by means of a screw thread.

### SUMMARY OF THE INVENTION

The plunger rod of the injection device, OptiPen®, corresponds to the rod-shaped output member of the present invention. To be able to move the plunger rod back to the rearmost position, the rod-shaped output member has grooves extending in lines in the axial direction on the outer circumference and, between the grooves are situated lines with a structured surface. In the illustrative embodiment described herein, this structured surface is a thread.

Furthermore, a guide element made up of at least two parts is provided, both parts of which are of annular shape and each have grooves extending in lines in the axial direction on their inner circumference and, between the grooves are situated lines with a structured surface. Here, the structured surface corresponds to an internal thread.

The problem on which the present invention is based is that of simplifying the handling of the device.

According to the invention, this is achieved by providing a clamp element which can be secured, releasably, on the rod-shaped output member, which clamp element, in the clamped state of the rod-shaped output member, is movable in order to set the dose to be injected or to dispense the dose, and which clamp element is automatically released upon release and removal of the cartridge.

### BRIEF DESCRIPTION OF THE FIGURES

An illustrative embodiment of the invention is shown in the drawing.

FIG. 1 shows a rod shaped output member and clamp element of the present invention.

### EMBODIMENTS OF THE INVENTION

An embodiment of the invention provides a device wherein, in a working position, the two parts of the guide element are turned relative to one another in such a way that the grooves of the one part are continued in the axial direction by the structured surface of the other part. This means that, in the working position, at least one of the two parts is always in engagement via its internal thread with the thread of the rod-shaped output member. In the working position, the two parts of the guide element are locked securely against rotating relative to one another.

An embodiment of the invention provides a device wherein, the rod-shaped output member is mounted securely against rotation in the injection device.

An embodiment of the invention provides a device wherein, before the dose quantity of an injection is set, the plunger is situated at a defined spacing from the cartridge. When an injection is triggered, the plunger is moved by a defined distance toward the cartridge. The spacing is dimensioned in such a way that the spacing corresponds exactly to the defined distance. Thus, if no dose is set, an insulin quantity of “0” is injected, because the defined distance corresponds exactly to the defined spacing and the plunger then bears precisely on the cartridge without having reduced the latter's volume.

An embodiment of the invention provides a device comprising, a rotatable setting element with which the dose quantity can be set. This is done, before the injection is triggered, by the plunger being moved toward the cartridge by a distance corresponding to the dose quantity. With the rotatable setting element, the already described guide element comprising the two parts is turned. As a result of the turning of the guide element and the rotationally fixed mounting of the rod-shaped output member, the rod-shaped output member is moved forward corresponding to the pitch of the thread.

An embodiment of the invention provides a device wherein the working position, the guide element can be turned relative to the rod-shaped output member by integral multiples of a defined angle. At the start of the turning, a first of the two parts is situated relative to the rod-shaped output element in a position in which the structured surface of the inner circumference of the one part is located opposite the grooves of the rod-shaped output member, and a second of the two parts is located relative to the rod-shaped output element in a position in which the structured surface of the inner circumference of the one part is located opposite the structured surface of the rod-shaped output member. This guarantees that a defined relative angle position is obtained between the two parts of the guide element and the rod-shaped output member.

An embodiment of the invention provides a device wherein angle position of the two parts of the guide element relative to one another is locked in the working position. This therefore ensures that in the working position at least one of the threads of the two parts is always in engagement with the thread of the rod-shaped output member.

An embodiment of the invention provides a device wherein the two parts of the guide element can be brought into an unlocked position. By means of a manual actuation by the user, the locking of the two parts relative to one another in the working position is canceled. Then, by means of an actuation by the user, the second part is turned relative to the first part so that the grooves of the one part are continued in the axial direction by the grooves of the other part. Based on the above-described definitions of the angle position of the first part relative to the rod-shaped output member, the structured surface of the two parts lies opposite the grooves of the rod-shaped output member in this unlocked position. The engagement of the threads of the two parts of the guide element with the thread of the rod-shaped output member is thus released. In this unlocked position, the rod-shaped output member is thus freely movable in the axial direction. By a suitable inclination of the injection device, it can thus fall back into the starting position.

An embodiment of the invention provides a device wherein the two parts of the guide element can be locked, for example, by means of the two parts having grooves extending along their outer circumference at corresponding angle positions, into which grooves rod-shaped elements can be pushed for locking. By removing the rod-shaped elements, the locking can be released.

An embodiment of the invention provides a device for guiding a rod-shaped output member (1) of an injection device with an exchangeable cartridge containing injection liquid, the rod-shaped output member (1) being able to be driven by defined distances in a direction reducing the volume of the cartridge in order to dispense the injection liquid, wherein a clamp element (2) is provided which can be secured releasably on the rod-shaped output member (1), which clamp element (2), in the clamped state of the rod-shaped output member (1), is movable in order to set the dose to be injected or to dispense the dose, and wherein the clamp element (2) is automatically released upon release and removal of the cartridge.

In other contexts, the operating principle of clamp elements of this kind is known, for example, from syringe guns used for dispensing silicone from cartridges. In these, the clamp element is moved forward by a defined distance. The rod-shaped output member then moves together with the clamp element. To return the rod-shaped output element, the clamp element can be released. The rod-shaped output element can then simply be moved back.

By means of the clamp element, it is therefore possible, during injection, to drive the rod-shaped output member by moving the clamp element, during injection, by the distance corresponding to the dose to be injected.

An embodiment of the invention provides a device according to the present invention, wherein at the time of injection the rod-shaped output member (1) is first arranged at a defined spacing from the cartridge, the rod-shaped output member (1) is then initially moved by means of the clamp element (2) in the direction of the cartridge, by a distance corresponding to the liquid to be injected, after which the injection is then carried out by moving the rod-shaped output member (1) a fixed distance in the direction of the cartridge.

An embodiment of the invention provides a device according to the present invention, wherein it is also possible, as in the prior art, to move the rod-shaped output element forward by a defined distance during injection, in which case the amount to be injected is set during the dose- setting, by correspondingly varying the spacing of the rod-shaped output element from the cartridge before injection. The clamp element is thus then situated in connection with the rod-shaped output element only during the dose-setting and is otherwise released from it.

An embodiment of the invention provides a device according to the present invention, wherein, at the time of injection, the rod-shaped output member is first arranged at a defined spacing from the cartridge, the rod-shaped output member is then initially moved by means of the clamp element in the direction of the cartridge, by a distance corresponding to the liquid to be injected, after which the injection is then carried out by moving the rod-shaped output member a fixed distance in the direction of the cartridge.

An embodiment of the invention provides a device according to the present invention, wherein, a further clamp element is provided which is released during setting of the dose and which, in order to carry out the injection, comes into engagement with the rod-shaped output member (1) and, in order to carry out the injection, is moved by a defined distance, and, while the injection is being carried out, the first clamp element is released, both clamp elements being released automatically upon release and removal of the cartridge.

An embodiment of the invention provides a device according to the present invention, wherein, a further clamp element is provided which is released during setting of the dose and which, in order to carry out the injection, comes into engagement with the rod-shaped output member and, in order to carry out the injection, is moved by a defined distance, and, while the injection is being carried out, the first clamp element is released, both clamp elements being released automatically upon release and removal of the cartridge.

In this way, it is easy to provide the possibility of setting the dose with a first clamp element and of carrying out the injection with a further clamp element, by means of these being brought into engagement at the corresponding times with the rod-shaped output element.

With the drive by means of the clamp element, a comparatively simple mechanical construction of the drive mechanism of an injection device is obtained, at the same time with simple handling when changing the cartridge. By virtue of the fact that the rod-shaped output member is not held by the clamp element or clamp elements when the cartridge is being changed, it can simply slip back by means of a suitable inclination of the injection device or can be pushed back as the new cartridge is inserted.

It is appreciated that certain features of the invention, which are, for clarity, described in the context of separate embodiments, may also be provided in combination in a single embodiment. Also, various features of the invention which are, for brevity, described in the context of a single embodiment, may also be provided separately or in any suitable sub-combination.

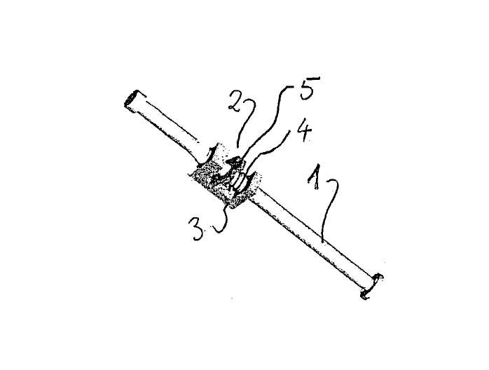

FIG. 1 shows a rod-shaped output member 1. A clamp element 2 can also be seen which has a support 3 which is made secure to a drive means in the housing of the injection device. It can also be seen how a clamp plate 3 with a spring 4 bears on the support 3 and thereby clamps the rod-shaped output member 1. By moving the support 3, the rod-shaped output member 1 is then also correspondingly moved.

As has been indicated above, a design is expedient in which, instead of the one clamp element shown, there are two clamp elements.

The present invention may be embodied in other specific forms without departing from the spirit or essential attributes thereof.

1. A device for guiding a rod-shaped output member (1) of a injection device with an exchangeable cartridge containing injection liquid, the rod-shaped output member (1) being able to be driven in defined distances in a direction reducing the volume of the cartridge in order to dispense the injection liquid, said device comprising a clamp element (2) which can be releasably secured on the rod-shaped output member (1), wherein the clamp element (2), in the clamped state of the rod-shaped output member (1), is movable in order to set the dose to be injected or to dispense the dose, and wherein the clamp element (2) is automatically released upon release and removal of the cartridge.

2. The device as claimed in claim 1, wherein at the time of injection the rod-shaped output member (1) is first arranged at a defined spacing from the cartridge, the rod-shaped output member (1) is then initially moved by means of the clamp element (2) in the direction of the cartridge, by a distance corresponding to the liquid to be injected, after which the injection is then carried out by moving the rod-shaped output member (1) a fixed distance in the direction of the cartridge.

3. The device as claimed in claim 2, wherein a further clamp element is provided which is released during setting of the dose and which, in order to carry out the injection, comes into engagement with the rod-shaped output member (1) and, in order to carry out the injection, is moved by a defined distance, and, while the injection is being carried out, the first clamp element is released, both clamp elements being released automatically upon release and removal of the cartridge.
